(19) United States
(12) Patent Application Publication
(10) Pub. No.: US 2026/0345356 A1
(43) Pub. Date: Nov. 5, 2026

(54) MANDREL FOR MANUFACTURING OF MEDICAL DEVICES

(71) Applicant: W. L. Gore & Associates, Inc., Newark, DE (US)

(72) Inventors: Robert Aochi, Morgan Hill, CA (US); John L. Fellows, Newark, DE (US); Jordan A. Hoof, Newark, DE (US); Herald Mullenaux, Mesa, AZ (US); Wade S. Nielsen, Phoenix, AZ (US); Colin J. Pemberton, Newark, DE (US); Thomas Rausch, Flagstaff, AZ (US); Chelsea Silberglied, Newark, DE (US); Justin W. Sokel, Flagstaff, AZ (US); Evan Thomason, Newark, DE (US); Dustin Williams, Newark, DE (US); Roark N. Wolfe, Flagstaff, AZ (US)

(21) Appl. No.: 19/660,559

(22) Filed: Apr. 28, 2026

Related U.S. Application Data
(60) Provisional application No. 63/797,840, filed on Apr. 30, 2025.

(51) Int. Cl. A61F 2/88

(52) U.S. Cl. CPC A61F 2/88; A61F 2240/001

(57) ABSTRACT

A mandrel for the manufacturing of medical devices, including a grooved portion having one or more grooves which may facilitate the alignment of a wire for the manufacture of a frame or stent and one or more apertures configured to pass air from an inner lumen of the mandrel to an ambient environment of the mandrel to facilitate the removal of the medical device from the mandrel.

### CROSS-REFERENCE TO RELATED APPLICATION

This application claims the benefit of Provisional Application No. 63/797,840, filed Apr. 30, 2025, which is incorporated herein by reference in its entirety for all purposes.

### BACKGROUND

Wound stents are widely used in medical devices designed to support and maintain the patency of bodily lumens. These stents may include a tubular structure formed by a continuous wire that is wrapped around a longitudinal axis, which may or may not form a helical configuration. The helical design allows for flexibility and conformability to the natural anatomy of bodily lumens while providing radial support to keep the vessel open.

Manufacturing wound stents presents its own set of challenges. The process typically involves forming the stent pattern, wrapping it around a mandrel, and then heat-setting or otherwise fixing the shape. Achieving precise and consistent geometries throughout the stent, especially in transition areas, can be difficult and may impact the efficiency of the manufacturing process.

Mandrels for such manufacture are generally known, including mandrels with grooves for seating wires and apertures for airflow around the mandrel and manufactured device for dispersing ambient gases.

### SUMMARY

A mandrel for the manufacturing of medical devices, including a grooved portion having one or more grooves forming at least one grooved geometric pattern which may facilitate the alignment of a wire for the manufacture of a frame or stent. The mandrel has one or more apertures which place an inner lumen of the mandrel in communication with an ambient environment of the mandrel and are configured to pass air from an inner lumen of the mandrel to at least the outside diameter of the mandrel to facilitate the removal of the medical device from the mandrel.

According to one example (“Example 1”), a vacuum mandrel for securing a support member to a cover member of a medical device is provided. The vacuum mandrel may include an elongate body having an outer surface with an optional grooved geometrical pattern formed into the outer surface. The grooved geometrical pattern optionally includes a circumferential, undulating path. The mandrel may include a vacuum lumen extending within the elongate body. The mandrel may optionally include a plurality of vacuum apertures in communication with the grooved geometrical pattern and the vacuum lumen.

According to another example (“Example 2”), a method of manufacturing a medical device is provided. The method may include positioning a cover member over a grooved portion of a mandrel. The grooved portion of the mandrel optionally includes one or more apertures configured to facilitate air flow. The method may optionally include applying a vacuum pressure to the cover member via the one or more apertures to draw the cover member into a groove defined within the grooved portion of the mandrel. The method may include inserting a length of a support member into the groove so that the cover member at least partially surrounds the support member. The method may optionally include coupling the support member to the cover member. The method may include applying positive pressure to the cover member via the one or more apertures to facilitate removal of the cover member and the length of the support member from the mandrel.

According to another example (“Example 3”), a method of manufacturing a vacuum mandrel is provided. The method may include forming a circumferential, undulating grooved geometrical pattern into an outer surface of an elongate body. The elongate body may optionally have a vacuum lumen extending within the elongate body. The method may optionally include forming a plurality of vacuum apertures in communication with the grooved geometrical pattern and the vacuum lumen within the elongate body.

In various examples, the circumferential path may optionally extend helically along the outer surface.

In various examples, the undulating pattern may optionally define a plurality of peaks and valleys. One or more vacuum apertures of the plurality of vacuum apertures may optionally be positioned in at least one of the plurality of peaks and valleys.

In various examples, the plurality of vacuum apertures may optionally be positioned entirely within one or more grooves of the grooved geometrical pattern.

In various examples, one or more grooves of the grooved geometrical pattern may optionally have a V-shaped cross-section.

In various examples, the vacuum mandrel may optionally include one or more marker indentations, the one or more marker indentations optionally being associated with at least one of the one or more grooves, the one or more marker indentations being optionally formed into the outer surface and defining a widened portion of the one or more grooves so that the one or more marker indentations have a greater width than adjacent portions of the one or more grooves. At least one of the one or more marker indentations may optionally be associated with an apex portion of the one or more grooves. At least one of the one or more marker indentations may optionally be associated with an intermediate portion of the one or more grooves located between an adjacent peak and valley of the one or more grooves. The one or more marker indentations may optionally have a length dimension that is angularly aligned to the adjacent portions of the one or more grooves. The one or more marker indentations may optionally have a length dimension that is angularly offset from the adjacent portions of the one or more grooves. The one or more marker indentations may optionally vary in depth along a longitudinal dimension of the groove. The one or more marker indentations may optionally be constant in depth along a longitudinal dimension of the groove. The one or more marker indentations may optionally have a transverse cross-sectional shape that corresponds to a truncated circle, rectangle, triangle, or trapezoid shape.

In various examples, the support member may optionally be a preformed stent arranged on the mandrel consistent with the groove of the grooved portion.

In various examples, the support member may optionally be a wire wound directly into the groove of the grooved portion.

In various examples, positioning the cover member over the grooved portion of the mandrel may optionally include wrapping a graft material over the grooved portion of the mandrel in a helical pattern.

In various examples, coupling the support member to the cover member may optionally include applying an adhesive over the support member positioned within the groove. The support member may optionally be fully surrounded by the cover member and the adhesive after coupling the support member to the cover member.

In various examples, the one or more apertures may optionally be positioned within the groove.

In various examples, a release coating may optionally be applied to the outer surface of the elongate body.

In various examples, the elongate body may optionally be electropolished.

In various examples, the elongate body may optionally be polished by tumbling.

In various examples, the elongate body may optionally be polished by vibratory finishing.

In various examples, the elongate body may optionally be polished by electrochemical processes.

In various examples, the elongate body may optionally be polished by mechanical buffing processes.

In various examples, the elongate body may optionally be polished by vibratory finishing.

In various examples, one or more grooves of the grooved geometrical pattern may optionally have a V-shaped cross-section.

In various examples, each vacuum aperture of the plurality of vacuum apertures may optionally be positioned entirely within one or more grooves of the grooved geometrical pattern.

The foregoing Examples are just that and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1A is a perspective view of a mandrel having a grooved portion in accordance with an embodiment, the mandrel having a tapered portion;

FIG. 1B is a perspective view of a mandrel having a grooved portion in accordance with an embodiment, the mandrel being generally straight;

FIG. 2 is a side and partial cross-sectional view of the mandrel of FIG. 1A at a first revolution point;

FIG. 3 is a close-up view of a pattern of grooved rows and apertures having a first aperture shape within the grooved portion of the mandrel of FIG. 1A;

FIG. 4A is a cross-section of a grooved row of FIG. 3 having a first depth;

FIG. 4B is a cross-section of a grooved row of FIG. 3 having a second depth;

FIG. 5A is a cross-section of a grooved row of FIG. 3 having a first cross-sectional shape;

FIG. 5B is a cross-section of a grooved row of FIG. 3 having a second cross-sectional shape;

FIG. 5C is a cross-section of a grooved row of FIG. 3 having a third cross-sectional shape;

FIG. 5D is a cross-section of a grooved row of FIG. 3 having a fourth cross-sectional shape;

FIG. 5E is a cross-section of a grooved row of FIG. 3 having a fifth cross-sectional shape;

FIG. 5F is a cross-section of a grooved row of FIG. 3 having a sixth cross-sectional shape;

FIG. 5G is a cross-section of a grooved row of FIG. 3 having a first asymmetrical cross-sectional shape;

FIG. 5H is a cross-section of a grooved row of FIG. 3 having a second asymmetrical cross-sectional shape;

FIG. 6A is a close-up view of a plurality of apertures having a second aperture shape;

FIG. 6B is a close-up view of a plurality of apertures having a third aperture shape;

FIG. 6C is a close-up view of a plurality of apertures having a fourth aperture shape;

FIG. 7 is an illustration of a first method for manufacturing a medical device using the mandrel of FIG. 1;

FIG. 8A is an illustration of a first medical device manufactured using the method of FIG. 7;

FIG. 8B is an illustration of a second medical device manufactured using the method of FIG. 7; and

FIG. 9 is an illustration of a second method for manufacturing a medical device using the mandrel of FIG. 1.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale but may be exaggerated to illustrate various aspects of the present disclosure, and in that regard, the drawing figures should not be construed as limiting.

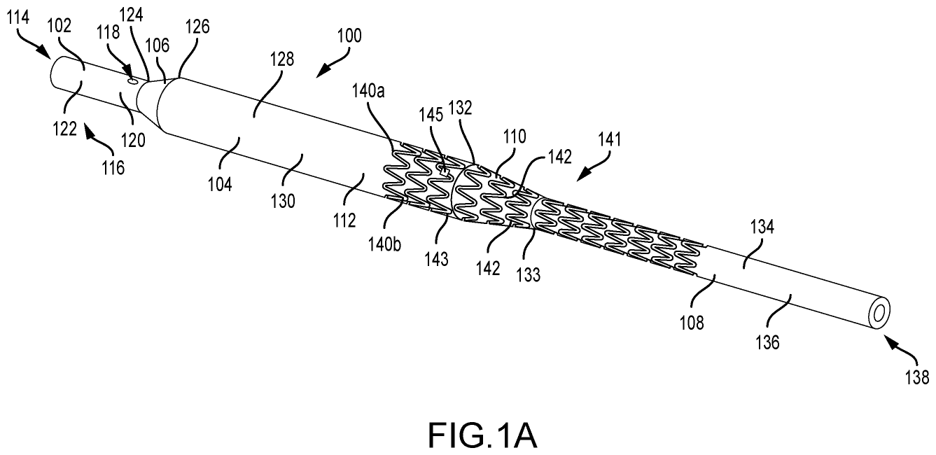

Referring to FIG. 1A, a mandrel 100 is illustrated. As shown, the mandrel 100 is a single piece, either formed by integral or permanently coupled components. However, in various examples mandrel 100 may include separate, connected and separable components (e.g., the mandrel 100 may be a multi-piece construct that is configured to be disassembled). As shown, the mandrel 100 (and more specifically, the working length of the mandrel over which a device, such as an endoprosthesis, is formed) includes one or more tapered portions (106, 110) and one or more cylindrical portions (102, 104, 108). For example, the mandrel 100 optionally includes an end portion 102, a first body portion 104, and a first tapered portion 106 extending from end portion 102 to first body portion 104. As shown, mandrel 100 may further include a second body portion 108 and a second tapered portion 110 extending from first body portion 104 to second body portion 108. End portion 102, first body portion 104, first tapered portion 106, second body portion 108, and second tapered portion 110 may be constructed to form a hollow elongate body 112 defining an inner lumen 114.

The various portions may be coupled to one another by being formed as separate, connected components (e.g., bonded, welded, or adhered) or coupled to one another by being integrally formed with one another (e.g., milled, additive manufactured, or cast). And, one or more portions may be formed as separate, connected components and one or more portions may be formed as integral components.

End portion 102 may be arranged at a proximal end 116 of mandrel 100 and include a vent hole 118 which places the ambient environment of mandrel 100 in fluid communication with inner lumen 114. End portion 102 includes an outer surface 120 defining a first outer diameter 122.

As mentioned above, first tapered portion 106 extends from end portion 102 to first body portion 104. First tapered portion 106 may have a proximal end 124 having an outer diameter consistent with first outer diameter 122 and a distal end 126 having an outer diameter consistent with an outer diameter of first body portion 104, as described further herein. In other words, the outer diameter of first tapered portion 106 may increase along a length of first tapered portion 106 from first outer diameter 122 to second outer diameter 128.

First body portion 104 is arranged distally adjacent to first tapered portion 106, extends from, and is coupled to first tapered portion 106. First body portion 104 includes an outer surface 130 defining a second outer diameter 128. As shown, second outer diameter 128 may be larger than first outer diameter 122. In other embodiments, first outer diameter 122 may be larger than second outer diameter 128 or consistent with second outer diameter 128. In such embodiments, the outer diameter of first tapered portion 106 may decrease from the first outer diameter to the second outer diameter or may otherwise be omitted.

As mentioned above, second tapered portion 110 extends from first body portion 104 to second body portion 108. Second tapered portion 110 is coupled to first body portion 104 and second body portion 108 as previously described.

Second tapered portion 110 may have a proximal end 132 having an outer diameter consistent with second outer diameter 128 and a distal end 133 having an outer diameter consistent with an outer diameter of second body portion 110. In other words, the outer diameter of second tapered portion 110 may decrease along a length of second tapered portion 110 from second outer diameter 128 to third outer diameter 134.

Second body portion 108 is arranged distally adjacent to second tapered portion 110 and is coupled to second tapered portion 110 as described above. Second body portion 108 includes an outer surface 135 defining third outer diameter 134. As shown third outer diameter 134 may be smaller than second outer diameter 128 and larger than first outer diameter 122.

In other embodiments, third outer diameter 134 may be larger than second outer diameter 128 and/or smaller than first outer diameter 122. In other embodiments, third outer diameter 134 may be consistent with second outer diameter 128 and/or first outer diameter 122. In embodiments in which third outer diameter 134 is consistent with second outer diameter 128, the outer diameter of second tapered portion 110 may be omitted. In embodiments in which third outer diameter 134 is larger than second outer diameter 128, the outer diameter of second tapered portion 110 may increase from the second outer diameter to the third outer diameter.

Second body portion 108 may terminate at a distal end 137 of mandrel 100. Distal end 137 may include a plug 139, which may be at least partially received within inner lumen 114 of mandrel 100. Plug 139 may be coupled to second body portion 108 via welding (e.g., laser), adhesives, fasteners, interference fit, or other means. Additionally, the plug 139 may be coupled to the second body portion 108 by being formed as an integral part of the distal end 137 (e.g., the lumen may be a blind hole, or lumen that terminates prior to the distal end 137 with the material of the distal end 137 forming the plug 139).

In some embodiments, as illustrated in FIG. 1B, mandrel 100 and/or elongate body 112 may not be tapered, or may have a constant outer diameter. For example, mandrel 100 may have a consistent diameter from proximal end 116 of mandrel 100 to a distal end 137 of mandrel 100 and/or may have a constant outer diameter for at least the working length of the mandrel 100 (i.e., the length corresponding to the portion of the mandrel 100 where a device is formed).

A grooved portion 141 may be formed on elongate body 112 as shown in FIGS. 1A-1B. As illustrated, grooved portion 141 includes one or more groove(s) defining a grooved geometrical pattern 143. As shown, grooved geometrical pattern 143 may include one or more groove(s) forming an undulating or wavy circumferential path defining peaks 140a and valleys 140b. Grooved portion 141 may extend from first body portion 104 into second body portion 108 and may cover the entirety of second tapered portion 110.

As shown, grooved geometrical pattern 143 may include a single connected groove extending along an undulating or wavy circumferential path in the outer surface, the path extending in a helical manner along a length of elongate body 112. In other embodiments, grooved geometrical pattern 143 may include a plurality of grooves following an undulating or wavy circumferential path in a similar helical manner. Still further, grooved geometrical pattern 143 may include one or more grooves that extend in a non-helical paths (e.g., circumferential or longitudinal paths). In the case of a grooved geometrical pattern 143 including multiple grooves, one or more of such grooves may be intersecting or non-intersecting.

As shown, grooved geometrical pattern 143 is defined by a plurality of rows 142, which may or may not be of a helical path. A distance between adjacent rows may vary or may be constant. For example, each row 142 may be equidistant from adjacent rows. In some embodiments, peaks 140a and/or valleys 140b may have a crest or trough height which is generally consistent along the entire or partial length of the undulating or wavy circumferential path or may vary in crest or trough height along the entire or partial length of the undulating or wavy circumferential path.

In some embodiments, marker indentations 145 for receiving gold markers, radiopaque markers, or other imaging indicators (e.g., fluoroscopic) may be associated with the grooved portion 141 (e.g., formed as widened part of the grooved portion 141) to facilitate positioning of components during medical device manufacturing as described further herein. Marker indentations 145 may have a greater width than adjacent portions of the groove(s) 138 within grooved portion 141. Such markers may be used to indicate various device features and/or orientations (e.g., a beginning and/or ending point of a circumference of elongate body 112, a proximal and/or distal end of grooved portion 141, device rotational orientation, or the like). Other uses for such indicators are also contemplated.

In some embodiments, marker indentations 145 may be associated with an apex portion of the groove(s) 138. In other embodiments, marker indentations 145 may be associated with an intermediate portion of groove(s) 138, e.g., between peaks 140a and valleys 140b of groove(s) 138. In some embodiments, marker indentations 145 may have a length dimension that is angularly aligned to the adjacent portions of groove(s) 138. In other embodiments, marker indentations 145 may have a length dimension that is angularly offset form the adjacent portions of groove(s) 138. In some embodiments, marker indentations 145 may vary in depth along a longitudinal dimension of groove(s) 138. In other embodiments, marker indentations 145 may have a constant depth along a longitudinal dimension of groove(s) 138. In some embodiments, marker indentations 145 may have a transverse cross-sectional shape that corresponds to a truncated circle, rectangle, triangle, or trapezoid shape. In some embodiments, marker indentations 145 varying in these features may be included.

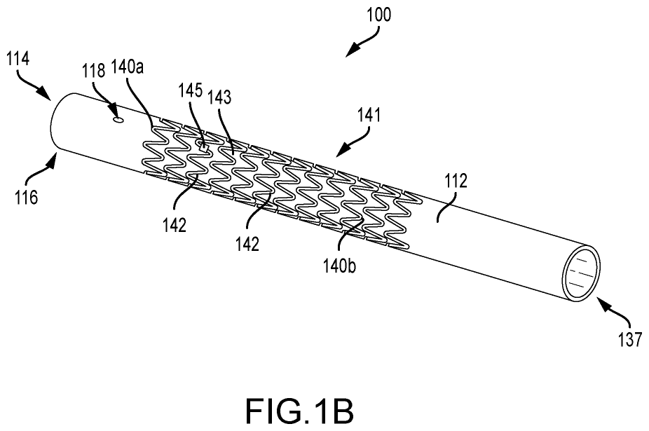

Now referring to FIG. 2, a plurality of apertures 144 may be formed within grooved portion 141. As shown, apertures 144 may be formed within groove(s) of grooved geometrical pattern 143 themselves and/or outside of the groove(s) of grooved geometrical pattern 143 within an outer surface of elongate body 112. In some embodiments, apertures 144 may be formed adjacent to groove(s) 138 (e.g., adjacent to a peak 140a or valley 140b of groove(s) 138 and/or in-between rows 142 of grooved portion 141. In other embodiments, apertures 144 may be spaced apart from grooved portion 141 along elongate body 112.

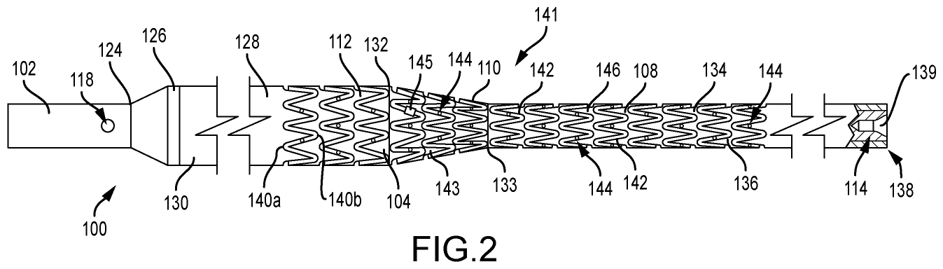

For example, referring to FIG. 3, apertures 144a may be positioned at an apex 147, e.g., a peak or valley, of rows 142 within groove(s) 138 and/or between apices 147 of rows 142 within groove(s) 138. Additionally or alternatively, apertures 144b may be positioned outside of groove(s) 138 between apices 147 of rows 142 and/or at another intermediate position between rows 142 outside of groove(s) 138. In some embodiments, one or more turns, or rows of the groove(s) has between 1 and 50 apertures 144B positioned within the grooves, for example, although any of a variety of different numbers of apertures are contemplated.

In various examples, there are between 1 and 50 apertures between at least two adjacent rows 142 defined by the groove(s) 138, although any of a variety of different numbers of apertures are contemplated. The various apertures 144B may be regularly spaced about a circumference of the mandrel 100 or may be at irregular intervals about one or more rows of the groove(s) 138. Additionally, the various apertures 144B may be regularly spaced along the length of the mandrel 100 or may be at irregular intervals along the length. In some examples, the apertures are regularly spaced about a helical path around the circumference of the mandrel 100 and along the length of the mandrel 100 for a plurality of the groove(s) 138.

Referring to FIG. 2, apertures 144 extend through a wall 146 of elongate body 112 so that each aperture 144 provides a fluid pathway between inner lumen 114 of mandrel 100 and an ambient environment of mandrel 100. In other words, air or gas may pass from inner lumen 114 into an ambient environment of mandrel 100 via apertures 144 to provide positive pressure exiting mandrel 100. Additionally, or alternatively, air or gas may pass from an ambient environment of mandrel 100 into inner lumen 114 via apertures 144. In other words, apertures 144 may serve as vacuum apertures for mandrel 100; in such embodiments, mandrel 100 may be referred to as a vacuum mandrel.

Grooved geometrical pattern 143, and more specifically the one or more groove(s), may be formed on elongate body 112 using etching, milling, or additive printing processes. In some embodiments, the width of the groove(s) may vary in size dependent on the size of the support member to be received within the groove(s). For example, the width of the groove(s) may be sized and shaped to receive a wire or other support member having a diameter of about 0.004 inches to about 0.040 inches. As such, the average width of the groove(s) may be about 0.001 inches to about 0.080 inches.

For example, the average width of the groove(s) may be about 0.001 inches, about 0.002 inches, about 0.003 inches, about 0.004 inches, about 0.005 inches, about 0.010 inches, about 0.015 inches, about 0.020 inches, about 0.025 inches, about 0.030 inches, about 0.035 inches, about 0.040 inches, about 0.045 inches, about 0.050 inches, about 0.055 inches, about 0.060 inches, about 0.065 inches, about 0.070 inches, about 0.075 inches, about 0.080 inches, or any measurement therebetween. In some embodiments, the average width of the groove(s) may be less than 0.001 inches or more than 0.080 inches.

In other words, the width of the groove(s) may be sized and shaped to receive a wire or other support member having a diameter of about 0.102 mm to about 1.016 mm. A such, the average width of the groove(s) may be about 0.030 mm to about 2.030 mm.

For example, the average width of the groove(s) may be about 0.025 mm, about 0.051 mm, about 0.076 mm, about 0.102 mm, about 0.127 mm, about 0.254 mm, about 0.381 mm, about 0.508 mm, about 0.635 mm, about 0.762 mm, about 0.889 mm, about 1.016 mm, about 1.143 mm, about 1.270 mm, about 1.397 mm, about 1.524 mm, about 1.651 mm, about 1.778 mm, about 1.905 mm, about 2.032 mm, or any measurement therebetween. In some embodiments, the average width of the groove(s) may be less than 0.025 mm or more than 2.032 mm.

In some embodiments, the width of the groove(s) may have a greater or lesser relationship to the width of the support member to be received to facilitate visibility, aid in placement of the support member, and/or to meet alignment specification requirements of the resulting device. In some embodiments, the maximum width, or the measured width, may be the width relative to the outer surface of the elongate body 112.

Grooved geometrical pattern 143 may be formed via, for example, an engraving cutter, additive manufacturing, a ball end mill, etching, or another method known in the art. For example, a ball end mill or engraving cutter with a tip having a diameter of between about 0.005 inches and about 0.100 inches may be used to form grooved geometrical pattern 143. For example, the ball end mill or engraving cutter tip may have a diameter of about 0.005 inches, about 0.010 inches, about 0.015 inches, about 0.020 inches, about 0.025 inches, about 0.030 inches, about 0.035 inches, about 0.040 inches, about 0.045 inches, about 0.050 inches, about 0.055 inches, about 0.060 inches, about 0.065 inches, about 0.070 inches, about 0.075 inches, about 0.080 inches, about 0.085 inches, about 0.090 inches, about 0.095 inches, about 0.100 inches, or any measurement therebetween.

The ball end mill, engraving cutter, additive manufacturing, etching, or other methods used to form grooved geometrical pattern 143 may depend on application of mandrel 100 due to the relationship between each method and the width and depth of the resultant groove(s); for example, the diameter of the ball end mill or the engraving cutter tip and the width and depth of the resultant groove(s) or another desired width and depth of groove(s) best attained by another method.

In some embodiments, the diameter of the ball end mill or engraving cutter tip may be less than 0.005 inches or more than 0.100 inches. In other words, in some embodiments, a ball end mill or engraving cutter tip having a diameter of between about 0.127 mm and about 2.54 mm may be used to form grooved geometrical pattern 143. For example, the ball end mill or engraving cutter tip may have a diameter of about 0.127 mm, about 0.254 mm, about 0.381 mm, about 0.508 mm, about 0.635 mm, about 0.762 mm, about 0.889 mm, about 1.016 mm, about 1.143 mm, about 1.270 mm, about 1.397 mm, about 1.524 mm, about 1.651 mm, about 1.778 mm, about 1.905 mm, about 2.032 mm, about 2.159 mm, about 2.286 mm, about 2.413 mm, about 2.54 mm, or any measurement therebetween. In some embodiments, the diameter of the ball end mill or engraving cutter tip may be less than 0.127 mm or more than 2.54 mm.

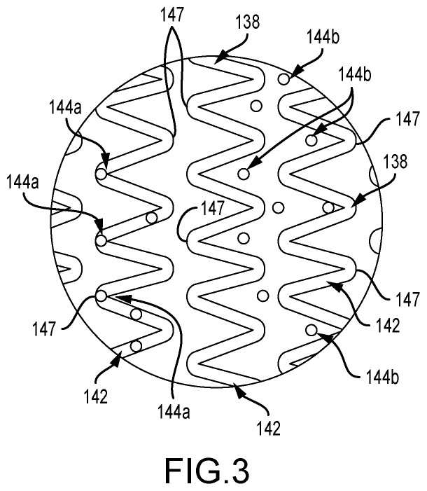

In some embodiments, the depth of the groove(s) may vary depending on the width of the support member to be received within the groove(s). For example, the depth of the groove(s) may be sized and shaped to receive a wire or other support member having a diameter of about 0.004 inches to about 0.040 inches. As such, the average depth of the groove(s) may be about 0.0010 inches to about 0.0500 inches. For example, as shown in comparison between the groove cross-sectional figures FIG. 4A and FIG. 4B, the groove(s) 138a may be shallow while the groove(s) 138b may be deep, dependent on the application of the mandrel.

For example, the average depth of the groove(s) may be about 0.0010 inches, about 0.0020 inches, about 0.0040 inches, about 0.0060 inches, about 0.0080 inches, about 0.0100 inches, about 0.0120 inches, about 0.0140 inches, about 0.0160 inches, about 0.0180 inches, about 0.0200 inches, about 0.0220 inches, about 0.0240 inches, about 0.0260 inches, about 0.0280 inches, about 0.0300 inches, about 0.0320 inches, about 0.0340 inches, about 0.0360 inches, about 0.0380 inches, about 0.0400 inches, about 0.0420 inches, about 0.0440 inches, about 0.0460 inches, about 0.0480 inches, about 0.0500 inches, or any measurement therebetween. In some embodiments, the depth of the groove(s) may be less than 0.0020 inches or more than 0.0500 inches.

In other words, in some embodiments, the depth of the groove(s) may be sized and shaped to receive a wire or other support member having a diameter of about 0.102 mm to about 1.016 mm. As such, the average depth of the groove(s) may be about 0.025 mm to about 1.270 mm.

For example, the average depth of the groove(s) may be about 0.025 mm, about 0.051 mm, about 0.102 mm, about 0.152 mm, about 0.203 mm, about 0.254 mm, about 0.305 mm, about 0.356 mm, about 0.406 mm, about 0.457 mm, about 0.508 mm, about 0.559 mm, about 0.610 mm, about 0.660 mm, about 0.711 mm, about 0.762 mm, about 0.813 mm, about 0.864 mm, about 0.914 mm, about 0.965 mm, about 1.016 mm, about 1.067 mm, about 1.118 mm, about 1.168 mm, about 1.219 mm, about 1.270 mm, or any measurement therebetween. In some embodiments, the depth of the groove(s) may be less than 0.051 mm or more than 1.270 mm.

In some embodiments, the depth of the groove(s) may have a greater or lesser relationship to the width of the support member to be received to facilitate visibility, aid in placement of the support member, and/or to meet alignment specification requirements of the resulting device.

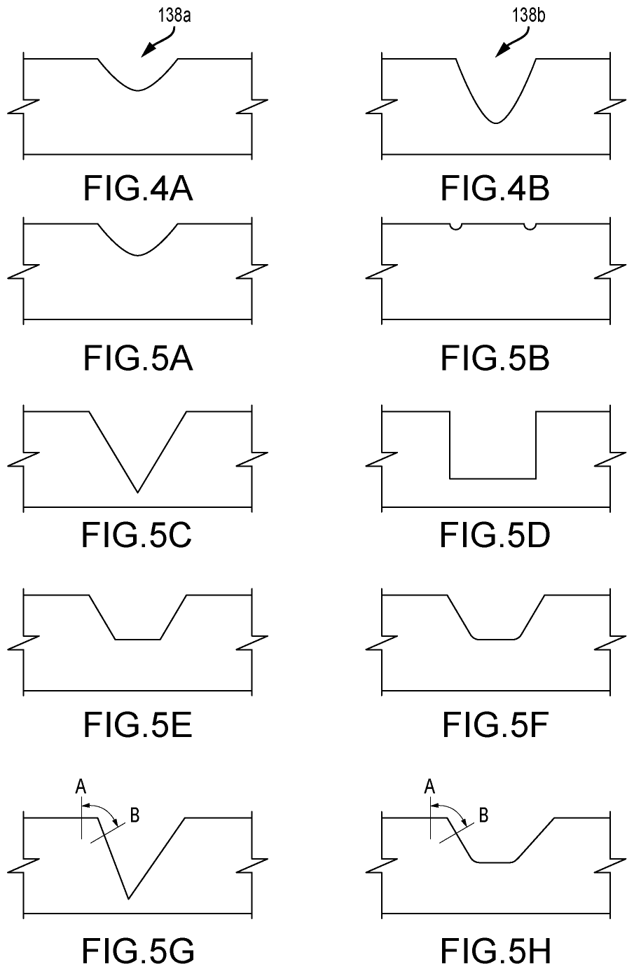

Grooved geometrical pattern 143 may have a semi-circular or semi-ovoid cross-sectional geometry (FIG. 5A) to facilitate reception of the one or more wires. In some embodiments, the cross-sectional geometry of grooved geometrical pattern 143 may vary. For example, grooved geometrical pattern 143 may have a triangular (FIG. 5B), V-shaped (FIG. 5C), squared (FIG. 5D), hexagonal (FIG. 5E), rounded hexagonal (FIG. 5F), or other cross-sectional geometry.

In some embodiments, the cross-sectional geometry of grooved geometrical pattern 143 may be generally symmetrical. In other embodiments, the cross-sectional geometry of grooved geometrical pattern 143 may be asymmetrical. For example, FIG. 5C illustrates a generally symmetrical V-shaped geometry, while FIG. 5G illustrates an asymmetrical V-shaped geometry.

The cross-sectional geometry of grooved geometrical pattern 143 may have a groove exit angle of between about 90° and about 160°. The “groove exit angle” as discussed herein is the angle between the outer surface of elongate body 112 and the surface of the groove—illustrated in FIGS. 5G and 5H as points A and B, respectively. The average groove exit angle may be about 90°, about 100°, about 110°, about 120°, about 130°, about 135°, about 140°, about 145°, about 150°, about 155°, about 160°, or any measurement therebetween. In some embodiments, e.g., the embodiment of FIG. 5C or 5F, the groove exit angle may be substantially the same on either side of the grooved geometrical pattern. In other embodiments, e.g., the embodiment of FIG. 5G or 5H, the groove exit angle may vary between each side of the grooved geometrical pattern.

The positioning of groove portion 136 and groove(s) of grooved geometrical pattern 143 relative to elongate body 112 may correspond with a desired geometry of the resulting medical device as described above. However, in some embodiments, the positioning of groove portion 136 and/or groove(s) of grooved geometrical pattern 143 may be offset from the desired geometry of the resulting medical device to account for shrinkage, stretch, and other natural variations in sizing of the graft and wire assembly during manufacture in order to meet certain device specifications.

The cross-sectional opening of apertures 144 may vary depending on device specifications. For example, the diameter of apertures 144 may be about 0.010 inches to about 0.200 inches. For example, the cross-sectional opening of apertures 144 may be about 0.010 inches, about 0.015 inches, about 0.020 inches, about 0.025 inches, about 0.030 inches, about 0.035 inches, about 0.040 inches, about 0.045 inches, about 0.050 inches, about 0.100 inches, about 0.150 inches, about 0.200 inches, or any measurement therebetween. In some embodiments, the cross-sectional opening of apertures 144 may be less than 0.010 inches or greater than 0.200 inches.

In other words, the diameter of apertures 144 may be about 0.254 mm to about 5.08 mm. For example, the cross-sectional opening of apertures 144 may be about 0.254 mm, about 0.381 mm, about 0.508 mm, about 0.635 mm, about 0.762 mm, about 0.889 mm, about 1.016 mm, about 1.143 mm, about 1.270 mm, about 2.54 mm, about 3.81 mm, about 5.08 mm, or any measurement therebetween. In some embodiments, the cross-sectional opening of apertures 144 may be less than 0.254 mm or greater than 5.08 mm.

In some embodiments, the cross-sectional opening of apertures 144 within a single mandrel 100 may be consistent. In other embodiments, the cross-sectional opening of apertures 144 within a single mandrel 100 may vary. As discussed herein the “cross-sectional opening” refers to the minor axis of the relative aperture.

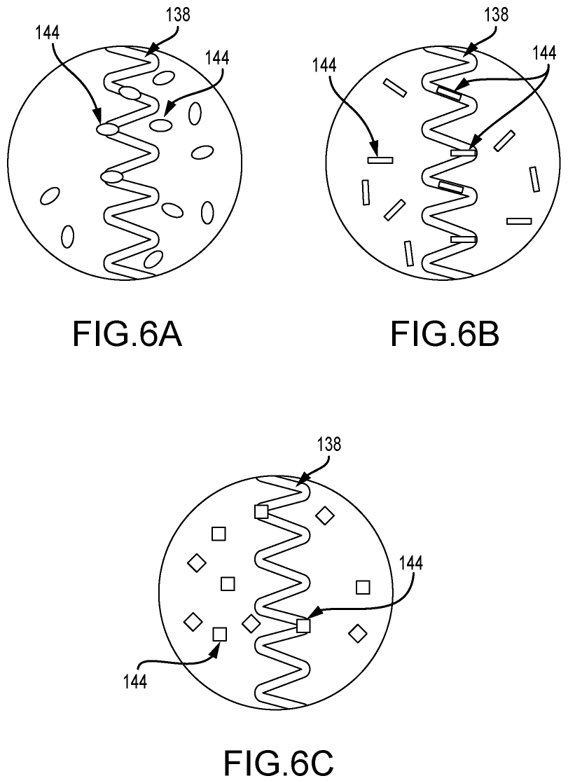

Apertures 144 may vary in shape. For example, referring briefly to FIG. 3, apertures 144a and/or 144b may have rounded shapes. In other embodiments, apertures 144 may be elongate (FIG. 6A), slit (FIG. 6B), square (FIG. 6C), or other various shapes. Apertures of any shape may be located within or without of groove(s) 138 as shown in FIGS. 3 and 6A-6B. Additionally, an aperture of any shape may be located entirely within groove(s) 138 as shown. Also, apertures of any shape may be greater in size than the width of groove(s) 138 so that the apertures may overflow one or more boundaries defined by groove(s) 138. As shown, apertures 144 may have various orientations relative to each other and/or groove(s) 138.

The number of apertures 144 per row 142, or revolution of grooved geometrical pattern 143, may vary depending on device specifications. For example, each row 142 may include about 5 apertures to about 30 apertures. For example, each row 142 may include about 5 apertures, about 10 apertures, about 15 apertures, about 20 apertures, about 25 apertures, about 30 apertures, or any number of apertures therebetween. In some embodiments, each row 142 may include less than 5 apertures or more than 30 apertures.

As discussed above, the outer diameter of mandrel 100 may vary in along the length of mandrel 100 in some embodiments. In other embodiments, the outer diameter of mandrel 100 may remain consistent along the length of mandrel 100. The diameter(s) of mandrel 100 may vary from mandrel to mandrel depending on device specifications. For example, the diameter(s) of mandrel 100 may be about 0.150 inches to about 2.000 inches.

For example, the diameter(s) of mandrel 100 may be about 0.150 inches, about 0.200 inches, about 0.250 inches, about 0.300 inches, about 0.350 inches, about 0.400 inches, about 0.450 inches, about 0.500 inches, about 0.550 inches, about 0.600 inches about 0.650 inches, about 0.700 inches, about 0.750 inches, about 0.800 inches, about 0.850 inches, about 0.900 inches, about 0.950 inches, about 1.000 inches, about 1.050 inches, about 1.100 inches, about 1.150 inches, about 1.200 inches, about 1.250 inches, about 1.300 inches, about 1.350 inches, about 1.400 inches, about 1.450 inches, about 1.500 inches, about 1.550 inches, about 1.600 inches, about 1.650 inches, about 1.700 inches, about 1.750 inches, about 1.800 inches, about 1.850 inches, about 1.900 inches, about 1.950 inches, about 2.000 inches, or any measurement therebetween. In some embodiments, the diameter(s) of mandrel 100 may be less than 0.150 inches or greater than 2.000 inches.

In other words, the diameter(s) of mandrel 100 may be about 3.81 mm to about 50.80 mm. For example, the diameter(s) of mandrel 100 may be about 3.81 mm, about 5.08 mm, about 6.35 mm, about 7.62 mm, about 8.89 mm, about 10.16 mm, about 11.43 mm, about 12.70 mm, about 13.97 mm, about 15.24 mm, about 16.51 mm, about 17.78 mm, about 19.05 mm, about 20.32 mm, about 21.59 mm, about 22.86 mm, about 24.13 mm, about 25.40 mm, about 26.67 mm, about 27.94 mm, about 29.21 mm, about 30.48 mm, about 31.75 mm, about 33.02 mm, about 34.29 mm, about 35.56 mm, about 36.83 mm, about 38.10 mm, about 39.37 mm, about 40.64 mm, about 41.91 mm, about 43.18 mm, about 44.45 mm, about 45.72 mm, about 46.99 mm, about 48.26 mm, about 49.53 mm, about 50.80 mm, or any measurement therebetween. In some embodiments, the diameter(s) of mandrel 100 may be less than 3.81 mm or greater than 50.80 mm.

Reasonable modifications to mandrel 100 are contemplated by the disclosure. For example, in some embodiments, elongate body 112 may be hollow as described above. In other embodiments, elongate body 112 may be solid with one or more surface features for feeding vacuum to the various groove(s) and/or apertures.

As discussed elsewhere herein, mandrel 100 may be generally straight, e.g., mandrel 100 may have a generally consistent diameter along a length of mandrel 100. In other embodiments, mandrel 100 may have a tapered diameter along one or more sections of mandrel 100 along a length of mandrel 100. For example, in some embodiments, mandrel 100 may have one or more tapered sections to connect one or more body sections, tip sections, or distal sections of mandrel 100 having varying diameters. In other embodiments, mandrel 100 may form an hour-glass shape. In yet other embodiments, mandrel 100 may form a dog bone shape.

In some embodiments, mandrel 100 may be bifurcated. In some embodiments, mandrel 100 may be formed of a single piece. In other embodiments, mandrel 100 may be formed of multiple pieces configured to fixedly or removably coupled together. In some embodiments, such removably attached pieces may be configured to be disassembled during the device manufacturing method described herein to facilitate removal of the one or more wires and/or the film and wire assembly upon completion.

In some embodiments, mandrel 100 may be configured to be used manually. In other embodiments, mandrel 100 may be configured to be installed within a larger machine or system. For example, mandrel 100 may include a proximal end and/or a distal end shaped and sized to interface with additional manufacturing equipment.

In some embodiments, mandrel 100 may be electropolished. In other embodiments, mandrel 100 may be subjected to abrasive media surface treatment. In other embodiments, mandrel 100 may be polished using electrochemical processes. In other embodiments, mandrel 100 may be polished using mechanical buffing processes. In other embodiments, the outer surface of mandrel 100 may be polished. In other embodiments, mandrel 100 may be tumbled. In other embodiments, mandrel 100 may be finished via vibratory finishing. In other embodiments, mandrel 100 may be provided with a coating. Such treatments may facilitate removal of a final device and/or graft and wire assembly upon completion of manufacture as described above.

In some embodiments, mandrel 100 may have varying diameters along length of mandrel 100 with a maximum diameter of about 8 mm to about 28 mm and a minimum diameter of about 8 mm to about 16 mm. For example, the maximum diameter may be about 4 mm, about 10 mm, about 12 mm, about 14 mm, about 16 mm, about 18 mm, about 20 mm, about 22 mm, about 24 mm, about 26 mm, about 28 mm or any measurement therebetween. The minimum diameter may be about 4 mm, about 5 mm, about 6 mm, about 8 mm, about 9 mm, about 10 mm, about 11 mm, about 12 mm, about 13 mm, about 14 mm, about 15 mm, about 16 mm, or any measurement therebetween. In some embodiments, mandrel 100 may have a maximum diameter that is less than 8 mm or greater than 20 mm. In some embodiments, mandrel 100 may have a minimum diameter that is less than 4 mm or greater than 16 mm.

In other embodiments, mandrel 100 may have a maximum diameter of about 44 mm to about 56 mm. For example, maximum diameter may be about 44 mm, about 46 mm, about 48 mm, about 50 mm, about 52 mm, about 54 mm, about 56 mm, or any measurement therebetween. In some embodiments, mandrel 100 may have a maximum diameter of less than 44 mm or greater than 56 mm. In various embodiments, the maximum diameter may be a consistent diameter along length of mandrel 100. In other embodiments, the maximum diameter may represent the greatest diameter of a plurality of diameters along length of mandrel 100.

In some embodiments, mandrel 100 may have a consistent diameter along length of mandrel 100, with a diameter of about 10 mm to about 28 mm. For example, the diameter may be about 10 mm, about 12 mm, about 14 mm, about 16 mm, about 18 mm, about 20 mm, about 22 mm, about 24 mm, about 26 mm, about 28 mm, or any measurement therebetween. In some embodiments, mandrel 100 may have a diameter that is less than 10 mm or greater than 28 mm.

In some embodiments, mandrel 100 may be configured to receive a support member having a diameter of about 0.0060 inches to about 0.0220 inches within grooved geometrical pattern 143. For example, mandrel 100 may be configured to receive a support member having a diameter of about 0.0060 inches, about 0.0080 inches, about 0.0100 inches, about 0.0120 inches, about 0.0140 inches, about 0.0160 inches, about 0.0180 inches, about 0.0200 inches, about 0.0220 inches, or any measurement therebetween. In some embodiments, mandrel 100 may be configured to receive a support member having a diameter less than 0.0060 inches or greater than 0.0220 inches.

In other words, in some embodiments, mandrel 100 may be configured to receive a support member having a diameter of about 0.152 mm to about 0.559 mm within grooved geometrical pattern 143. For example, mandrel 100 may be configured to receive a support member having a diameter of about 0.152 mm, about 0.203 mm, about 0.254 mm, about 0.305 mm, about 0.356 mm, about 0.406 mm, about 0.457 mm, about 0.508 mm, about 0.559 mm, or any measurement therebetween. In some embodiments, mandrel 100 may be configured to receive a support member having a diameter less than 0.152 mm or greater than 0.559 mm.

A biocompatible material for the cover member, graft, or film materials, discussed herein, may be used. In certain instances, the cover member, graft, or film may include a fluoropolymer, such as a polytetrafluoroethylene (PTFE) polymer or an expanded polytetrafluoroethylene (ePTFE) polymer. In some instances, the cover member, graft, or film may be formed of, such as, but not limited to, a polyethylene, an expanded polyethylene, a polyester, a silicone, a urethane, a polyethylene terephthalate, a polyetheretherketone, a polyaryletherketone, a polyetherketoneketone, a polyetherketone, a polyketone, a polyetheretherketoneketone, a polyetherketoneetherketoneketone, or another biocompatible polymer, including thermoplastics, or combinations thereof. In some instances, bioresorbable or bioabsorbable materials may be used, for example a bioresorbable or bioabsorbable polymer. In some instances, the cover member, graft, or film can include Dacron, polyolefins, carboxy methylcellulose fabrics, polyurethanes, or other woven, non-woven, or film elastomers.

In addition, nitinol (NiTi) may be used as the material of the support member, including a wire, frame, or stent (and any wire discussed herein), but other materials such as, but not limited to, stainless steel, L605 steel, polymers, MP35N steel, polymeric materials, Pyhnox, Elgiloy, or any other appropriate biocompatible material, and combinations thereof, can be used as the material of the support member. The super-elastic properties and softness of NiTi may enhance the conformability of the support member and resulting medical device. In addition, NiTi can be shape-set into a desired shape. That is, NiTi can be shape-set so that the resulting medical device tends to self-expand into a desired shape when the resulting medical device is unconstrained, such as when said medical device is deployed out from a delivery system.

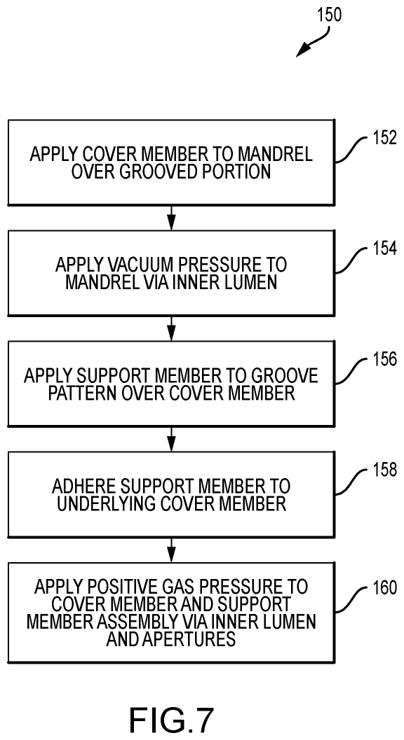

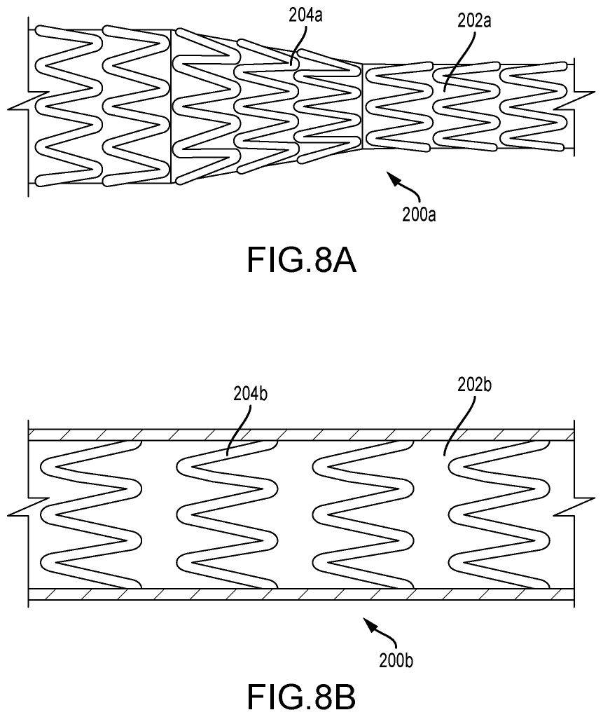

Now referring to FIG. 7 in view of FIGS. 1A-6C, a method 150 for manufacturing a medical device 200a, 200b (FIGS. 8A, 8B) using mandrel 100 is illustrated. For example, at step 152, a cover member 202a, 202b (FIGS. 8A, 8B) may be applied to mandrel 100 over grooved portion 141. In some processes, a preformed cover member, such as a graft construct may be applied over grooved portion 141. In other embodiments, cover member material, such as graft material, may be wrapped over grooved portion 141 so that the cover member is formed on mandrel 100. For example, the cover member material may be helically wrapped over grooved portion 141. In other embodiments, the cover member material may be circumferentially wrapped, e.g., similar to a cigarette wrapper, or otherwise positioned over grooved portion 141.

At step 154, a vacuum pressure is applied to mandrel 100 via inner lumen 114, e.g., a vacuum lumen 114. For example, mandrel 100 may be operatively coupled to a device configured to apply a vacuum pressure, so that vacuum pressure is applied to inner lumen 114 via, for example, vent hole 118 of end portion 102. The vacuum pressure may be applied to cover member 202a, 202b (FIGS. 8A, 8B) via apertures 144 so that cover member 202a, 202b (FIGS. 8A, 8B) is pulled into groove(s) 138 due to the positioning of apertures 144 and their fluidic connection to inner lumen 114. The application of vacuum pressure to the vacuum member facilitates a visual of and provides access to grooved geometrical pattern 143 over cover member 202a, 202b (FIGS. 8A, 8B).

In some embodiments, other forces may be used to position cover member 202a, 202b (FIGS. 8A, 8B) within grooved geometrical pattern 143, such as the use of a compression wrap, shrink tubing, physical wrap, stylus, roller, or hard surface.

At step 156, a support member 204a, 204b (FIGS. 8A, 8B) is applied to the grooved geometrical pattern 143 over cover member 202a, 202b (FIGS. 8A, 8B). As used herein, “support member” may include a length of wire, including one or more wires, that is formed into an undulating, helical shape by wrapping the wire around mandrel 100 according to grooved geometrical pattern 143. In other embodiments, “support member” may include a preformed device or frame, such as a stent and/or one or more discrete stent rings, in which embodiment the support member is positioned over grooved portion 141 and aligned with the grooved geometrical pattern 143. Support member 204a, 204b (FIGS. 8A, 8B) may be seated within the groove(s) of grooved geometrical pattern 143 via simple winding or positioning. In other embodiments, support member 204a, 204b (FIGS. 8A, 8B) may be forced into the groove(s) of grooved geometrical pattern 143 using a mechanical force, e.g., compression wrap, shrink tubing, physical wrap, stylus, roller, hard surface, or gas pressure. Upon completion of step 156, support member 204a, 204b (FIGS. 8A, 8B) may be circumferentially surrounded on at least three general sides, e.g., about 75% of the circumference of support member 204 a, 204 b (FIGS. 8A, 8B) may be surrounded by cover member 202a, 202b (FIGS. 8A, 8B).

At step 158, support member 204a, 204b (FIGS. 8A, 8B) is adhered to the underlying cover member 202a, 202b (FIGS. 8A, 8B) using adhesive (such as an adhesive coating, film, and/or tape), physical ties, a wrap or sleeve, or another constraining mechanism known in the art. In some embodiments, when appropriate, step 158 may also include heating the assembly to promote bonding between support member 204a, 204b (FIGS. 8A, 8B), cover member 202a, 202b (FIGS. 8A, 8B), and the constraining mechanism used. At the end of step 158, support member 204a, 204b (FIGS. 8A, 8B) may be completely surrounded along at least a partial length of support member 204a, 204b (FIGS. 8A, 8B). In other words, support member 204a, 204b (FIGS. 8A, 8B) may be fully encapsulated by cover member 202a, 202b (FIGS. 8A, 8B) and the constraining mechanism used.

At step 160, an outward gas pressure or positive pressure may be applied to inner lumen 114. For example, air or gas may be introduced into inner lumen 114 to raise the pressure within inner lumen 114. The positive air pressure is then forced through apertures 144, pushing resulting medical device 200a, 200b (FIGS. 8A, 8B) away from grooved geometrical pattern 143 and mandrel 100, facilitating removal of resulting medical device 200a, 200b (FIGS. 8A, 8B) from mandrel 100.

In some embodiments, such positive pressure may not be used for removal of the resulting medical device. In such embodiments, release of vacuum pressure may be completed before removal of the resulting medical device, but positive pressure may not be applied. In other embodiments, for example, in embodiments where vacuum pressure is not applied, the resulting medical device may be removed from the mandrel upon or before completion of step 158.

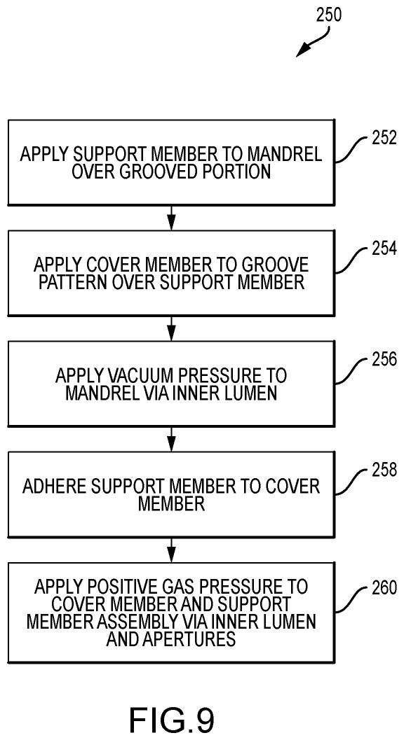

Now referring to FIG. 9 in view of FIGS. 1A-6C, a method 250 for manufacturing a medical device 200a, 200b (FIGS. 8A, 8B) using mandrel 100 is illustrated. For example, at step 252, a support member 204a, 204b (FIGS. 8A, 8B) may be applied to the grooved geometrical pattern 143. Support member 204a, 204b (FIGS. 8A, 8B) may be seated within the groove(s) of grooved geometrical pattern 143 via simple winding or positioning. In other embodiments, support member 204a, 204b (FIGS. 8A, 8B) may be forced into the groove(s) of grooved geometrical pattern 143 using a mechanical force, e.g., compression wrap, shrink tubing, physical wrap, stylus, roller, hard surface, or gas pressure.

At step 254, a cover member 202a, 202b (FIGS. 8A, 8B) may be applied to mandrel 100 over grooved portion 141 and over support member 204a, 204b (FIGS. 8A, 8B).

At step 256, a vacuum pressure may be applied to mandrel 100 via inner lumen 114, e.g., a vacuum lumen 114. For example, mandrel 100 may be operatively coupled to a device configured to apply a vacuum pressure, so that vacuum pressure is applied to inner lumen 114 via, for example, vent hole 118 of end portion 102. The vacuum pressure may be applied to cover member 202a, 202b (FIGS. 8A, 8B) via apertures 144 so that cover member 202a, 202b (FIGS. 8A, 8B) is pulled into a snug fit with seated support member 204a, 204b (FIGS. 8A, 8B) due to the positioning of apertures 144 and their fluidic connection to inner lumen 114.

At step 258, support member 204a, 204b (FIGS. 8A, 8B) is adhered to overlaying cover member 202a, 202b (FIGS. 8A, 8B) using, for example, heating to promote bonding between support member 204a, 204b (FIGS. 8A, 8B) and cover member 202a, 202b (FIGS. 8A, 8B).

At step 260, an outward gas pressure or positive pressure may be applied to inner lumen 114. For example, air or gas maybe introduced into inner lumen 114 to raise the pressure within inner lumen 114. The positive air pressure is then forced through apertures 144, pushing resulting medical device 200a, 200b (FIGS. 8A, 8B) away from grooved geometrical pattern 143 and mandrel 100, facilitating remove of resulting medical device 200a, 200b (FIGS. 8A, 8B) from mandrel 100.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

What is claimed is:

1. A vacuum mandrel for securing a support member to a cover member of a medical device, the vacuum mandrel including:
an elongate body having an outer surface with a grooved geometrical pattern formed into the outer surface, the grooved geometrical pattern including a circumferential, undulating path;
a vacuum lumen extending within the elongate body; and
a plurality of vacuum apertures in communication with the grooved geometrical pattern and the vacuum lumen.

2. The vacuum mandrel of claim 1, wherein the circumferential, undulating path extends helically along the outer surface.

3. The vacuum mandrel of claim 1, wherein the circumferential, undulating path defines a plurality of peaks and valleys.

4. The vacuum mandrel of claim 3, wherein one or more vacuum apertures of the plurality of vacuum apertures are positioned in at least one of the plurality of peaks and valleys.

5. The vacuum mandrel of claim 1, wherein the plurality of vacuum apertures are positioned entirely within one or more grooves of the grooved geometrical pattern.

6. The vacuum mandrel of claim 1, wherein one or more grooves of the grooved geometrical pattern has at least one of a V-shaped cross-section, a semi-circular cross-section, a rectangular cross-section, and a hexagonal cross-section.

7. The vacuum mandrel of claim 1, further including one or more marker indentations, the one or more marker indentations being associated with the at least one of the one or more grooves, the one or more marker indentations being formed into the outer surface and defining a widened portion of the one or more grooves so that the one or more marker indentations have a greater width than adjacent portions of the one or more grooves.

8. The vacuum mandrel of claim 7, wherein at least one of the one or more marker indentations is associated with an apex portion of the one or more grooves.

9. The vacuum mandrel of claim 7, wherein at least one of the one or more marker indentations is associated with an intermediate portion of the one or more grooves located between an adjacent peak and valley of the one or more grooves.

10. The vacuum mandrel of claim 7, wherein the one or more marker indentations has a length dimension that is angularly aligned to the adjacent portions of the one or more grooves.

11. The vacuum mandrel of claim 7, wherein the one or more marker indentations has a length dimension that is angularly offset from the adjacent portions of the one or more grooves.

12. The vacuum mandrel of claim 7, wherein the one or more marker indentations varies in depth along a longitudinal dimension of the groove.

13. The vacuum mandrel of claim 7, wherein the one or more marker indentations is constant in depth along a longitudinal dimension of the groove.

14. The vacuum mandrel of claim 7, wherein the one or more marker indentations has a transverse cross-sectional shape that corresponds to a truncated circle, rectangle, triangle, or trapezoid shape.

15. A method of manufacturing a medical device, including:
positioning a cover member over a grooved portion of a mandrel, the grooved portion of the mandrel including one or more apertures configured to facilitate air flow;
applying a vacuum pressure to the cover member via the one or more apertures to draw the cover member into a groove defined within the grooved portion of the mandrel;
inserting a length of a support member into the groove so that the cover member at least partially surrounds the support member;
coupling the support member to the cover member; and
removing the cover member and the length of the support member from the mandrel.

16. The method of claim 15, wherein the step of removing the cover member and the length of the support member from the mandrel includes applying positive pressure to the cover member via the one or more apertures.

17. The method of claim 15, wherein the support member is a preformed stent arranged on the mandrel consistent with the groove of the grooved portion.

18. The method of claim 15, wherein the support member is a wire wound directly into the groove of the grooved portion.

19. The method of claim 15, wherein the step of positioning the cover member over the grooved portion of the mandrel includes wrapping a graft material over the grooved portion of the mandrel in a helical pattern.

20. The method of claim 15, wherein the step of positioning the covet member over the grooved portion of the mandrel includes circumferentially wrapping a graft material over the grooved portion of the mandrel.

21. The method of claim 15, wherein the step of coupling the support member to the cover member may include applying an adhesive over the support member positioned within the groove.

22. The method of claim 21, wherein the support member is fully surrounded by the cover member and the adhesive after coupling the support member to the cover member.

23. The method of claim 15, wherein the one or more apertures are positioned within the groove.

24. A method of manufacturing a vacuum mandrel, the method including:
forming a circumferential, undulating grooved geometrical pattern into an outer surface of an elongate body, the elongate body having a vacuum lumen extending within the elongate body; and
forming a plurality of vacuum apertures in communication with the grooved geometrical pattern and the vacuum lumen within the elongate body.

25. The method of claim 24, further including applying a release coating to the outer surface of the elongate body.

26. The method of claim 24, further including polishing the elongate body by tumbling.

27. The method of claim 24, further including electropolishing the elongate body.

28. The method of claim 24, further including polishing the elongate body by electrochemical processes.

29. The method of claim 24, further including polishing the elongate body by mechanical buffing processes.

30. The method of claim 24, further including polishing the elongate body by vibratory finishing.

31. The method of claim 15, wherein one or more grooves of the grooved geometrical pattern has at least one of a V-shaped cross-section, a semi-circular cross-section, a rectangular cross-section, and a hexagonal cross-section.

32. The method of claim 15, wherein each vacuum aperture of the plurality of vacuum apertures are positioned entirely within one or more grooves of the grooved geometrical pattern.
